(19) United States
(12) Patent Application Publication
(10) Pub. No.: US 2013/0092709 A1
(43) Pub. Date: Apr. 18, 2013

(54) MEDICAMENT DISPENSING DEVICE

(71) Applicants: Isaac Salem, Brooklyn, NY (US); Elana Salem, Brooklyn, NY (US)

(72) Inventors: Isaac Salem, Brooklyn, NY (US); Elana Salem, Brooklyn, NY (US)

(73) Assignees: Elana Salem, Brooklyn, NY (US); Isaac Salem, Brooklyn, NY (US)

(21) Appl. No.: 13/650,547

(22) Filed: Oct. 12, 2012

Related U.S. Application Data
(60) Provisional application No. 61/546,312, filed on Oct. 12, 2011.

(51) Int. Cl. B65D 88/54; G01F 11/00

(52) U.S. Cl. 222/326; 222/390; 222/386

(57) ABSTRACT

A medicament dispensing device has a case with an upper portion, a lower portion, and a central portion. The device also has a plunger made up of a head and a stem. The head of the plunger is configured to contact a cartridge containing medicament that is receivable within the central portion of the case. The device also has a dosing assembly coupled to the stem and disposed partially within the case. The dosing assembly is selectively inhibited by a control system that limits the displacement of the dosing assembly and plunger within the case. The device also has a customizable dispensing assembly coupled to the upper portion and configured to receive medicament from the cartridge upon application of force from the plunger.

This application claims the benefit of U.S. Provisional Patent Application Ser. No. 61/546,312 filed Oct. 12, 2011, the entire disclosures of which are hereby incorporated by reference in this application in their entirety.

### TECHNICAL FIELD OF THE INVENTION

The present invention relates to a medicament dispensing device with dosing dial for administering dosages of oral medications. A dosing chart and a child safety lock may optionally be incorporated.

### BACKGROUND

Current devices which measure and administer liquid oral medications are very difficult to use and lead to dangerous inaccurate dosing, improper administering of the medicine, messy administration, and great inconvenience to both the user and the recipient.

There is a need for variation in drug-delivery attachments to accommodate the needs, comfort, and convenience of the administering individual and the recipient.

The typical dropper requires extraordinary time and effort by applying the proper amount of pressure on the rubber bulb on the top to extract the desired amount of liquid. This is frustrating to parents and leads to inaccurate dosing. In addition, it is difficult to regulate the flow of the medicament to the infant when using a dropper. This can lead to the infant aspirating on the medicament or the medicament dripping out of the infant's mouth. The syringe has similar problems to the dropper.

For example, oral syringes are problematic since they are difficult to control the flow of medicament, involve air bubbles forming within the liquid chamber and thereby lead to incorrect dosing, and are not child friendly.

Current medicine dispensation to infants and young children lacks the type of precision that is critical, comfortable, and convenient when delivering foreign substances into the young metabolisms and bodies of children recipients. In addition these devices do not have any child safety mechanism which prevents dispensing by a child. Other vessels which are used to store oral medicaments do not have any dosing or administering mechanism whatsoever and can lead to over and under dosing.

The dosing device of U.S. Pat. No. 8,007,476 provides for a piston-type dispensation mechanism for controlled dispensation of medicament from an ampoule attached to the piston housing. While U.S. Pat. No. 8,007,476 refers to cannula and nozzle administration, it fails to provide the public any details of how to use the piston-type dispensation mechanism in conjunction with precise oral administration design.

### SUMMARY OF THE INVENTION

Current droppers, syringes, and other vessels for dosing and administering medicine require skill and patience to deliver an accurate dose to the patient. The proposed invention offers precise dosing to the patient and convenience to the user.

The invention solves the issues in the prior art by providing a dosing regimen that can be accurately measured by the dosing mechanism. Dispensing the dose is modulated by only dispensing a “click at a time”. The child safety lock button prevents dispensing by a child. In addition, the dispensing of oral medicaments in cartridges which can be loaded into the proposed device will have less of a carbon footprint thereby having a potential environmental benefit.

Current devices which measure and administer liquid oral medications to children are very difficult to use and lead to dangerous inaccurate dosing, improper administering of the medicine, messy administration, and great inconvenience. The invention is a device which allows the accurate dosing of the medication via a dosing dial and push button mechanism. Interchangeable drug delivery attachments are included for the comfort and convenience of the parent and child. In addition, an optional pull-out dosing chart is incorporated into the device for the user's reference.

A device comprises a case having an upper portion, a lower portion, and a central portion, a plunger having a head and a stem, the head of the plunger disposed between the central portion and the lower portion, and the stem disposed in the lower portion, wherein the head is configured to contact a cartridge containing medicament receivable within the central portion upon application of force to the stem, a dosing assembly coupled to the stem and disposed partially within the case, the dosing assembly comprising a control system for selectively inhibiting displacement of the dosing assembly and plunger by precluding displacement of the dosing assembly within the case, and, a customizable dispensing assembly coupled to the upper portion and configured to receive medicament from the cartridge upon application of force from the stem, the dispensing assembly configured to receive the medicament through an opening from the central portion to the upper portion.

A method of administering medicament, comprises the steps of coupling a dispensing tip to a case based on a medicament to be dispensed, setting a control system to inhibit displacement of a dosing assembly within the case containing a cartridge of the medicament to be dispensed, the cartridge being disposed beneath the dispensing tip, pressing the dosing assembly while it is coupled to a stem of a plunger which is disposed partially within the case, displacing the plunger so that it displaces the bottom of the cartridge toward the dispensing tip and thereby causes the cartridge to provide medicament from the cartridge and into the dispensing tip, dispensing medicament from the dispensing tip, and inhibiting further displacement of the plunger by the control system.

The device and method may be implemented with a chart attached to the case to serve as a reference for the precision medicament administration according to the inventive embodiments described herein.

### BRIEF DESCRIPTION OF THE DRAWINGS

FIG. 1 shows an exemplary view of a medicament dispensing device.

FIG. 2A shows an exemplary cross-sectional view of a medicament dispensing device in an inactive configuration.

FIG. 2B shows an exemplary cross-sectional view of a medicament dispensing device in a dispensing configuration.

FIG. 2C shows another exemplary cross-sectional view of a medicament dispensing device in a dispensing configuration.

FIG. 3 shows another exemplary view of a medicament dispensing device.

FIGS. 4A-4D show exemplary views of medicament dispensing tips.

In the drawings like characters of reference indicate corresponding parts in the different figures.

### DETAILED DESCRIPTION

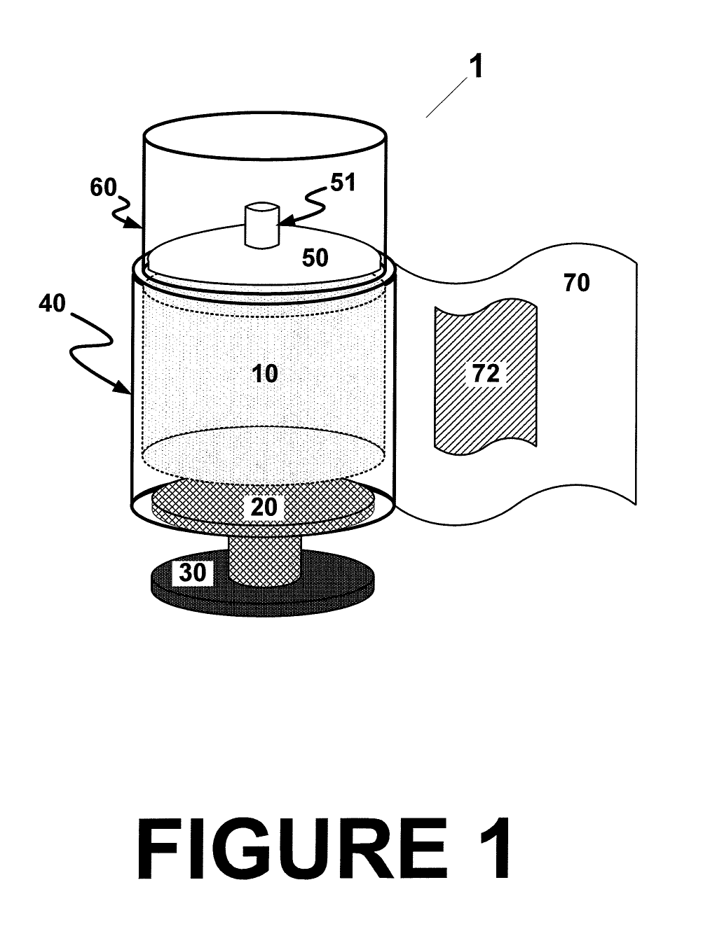

With reference to FIG. 1, an exemplary medicament dispensing device 1 includes a medicament cartridge 10, a plunger mechanism 20, a dosing mechanism 30, a case 40, a medicament dispenser 50, and a cap 60. Optionally, a covering 70 may be installed on or within case 40 for use in dosing and operating medicament dispensing device 1.

An exemplary medicament cartridge 10 may be any suitably shaped container for holding medicine to be dispensed. Preferably, cartridge 10 is a cylindrical container which contains an oral suspension of desired medicament.

An exemplary plunger mechanism 20 may be a mechanism that advances the bottom of cartridge 10 towards the top of the inside of case 40. Plunger mechanism 20 applies pressure on the bottom of cartridge 10 to allow dispensation of medicine stored therein. The amount of pressure supplied on cartridge 10 by plunger mechanism 20 may be controlled by dosing mechanism 30.

An exemplary dosing mechanism 30 may include a rotating arm that fits inside the arm of plunger mechanism 20 and/or case 40. By revolving dosing mechanism 30 into or away from plunger mechanism 20, dosing mechanism 30 may be set to a prescribed dosing arrangement. Dosing mechanism 30 may also include a child safety lock button which would need to be depressed to allow dosing of medicament from device 1.

An exemplary case 40 may be shaped to fit medicament cartridge 10, plunger mechanism 20, and dosing mechanism 30 so as to be operable according to the numerous embodiments disclosed herein. While case 40 is shown as a single construct, an exemplary case 40 may be modular depending on the size of the components, for example, case 40 may be divided into a dispensing section component that houses plunger mechanism 20 and dosing mechanism 30, a medicine holder component that houses cartridge 10, and a delivery component which may accommodate one or more types of dispensing tips 50 and/or delivery cannula channels 53 and 52. In a preferred embodiment, case 40, in either modular or single-bodied form, is cylindrical in shape.

An exemplary medicament dispenser 50 may be an interchangeable construct which can be snapped, screwed, twisted, or otherwise locked onto receiving portions in case 40. The interchangeability of medicament dispenser 50 allows for different shaped and sized tips, and for easy cleaning of the medicament dispensing components, such as, for example, the tip portion 51. The tip 51 may be molded in different colors, themes, and shapes for dispensing medicament to a recipient, such as, for example, a child, an infant, or an elderly person. Tip 51 preferably induces easy flow and administering of medicament from cartridge 10. An exemplary medicament dispensing tip 51 may be in the shape of a cup to allow a user to measure the dose in another vessel, such as, for example, a spoon, before administration to the recipient.

An exemplary cap 60 may be configured to safely cover all forms of case 40 and medicament dispenser 50 combinations. Cap 60 may be snapped onto case 40, twisted on top of case 40, or any other form of attachment to case 40 known to those skilled in the art. Alternatively, cap 60 may be attached to dispenser 50 and/or 51.

An exemplary covering 70 may be a dosing chart which can be used in the space between the cartridge 10 and case 40. Covering 70 may be attached to the outer surface of case 40 so as to wrap about the outside of case 40. Accordingly, covering 70 may have one or more adequately shaped transparent portions 72 that provide visibility to cartridge 10 and/or medicament within cartridge 10 to monitor replacement of cartridge 10 when covering 70 is not unraveled. Covering 70 may be a retractable plastic sheet conformed to wrap around case 40 when not in a fully extended state. Fully extended, covering 70 may possess medicinal information, including, but not limited to, dosing information for one or more medicaments in a convenient easy to read chart format.

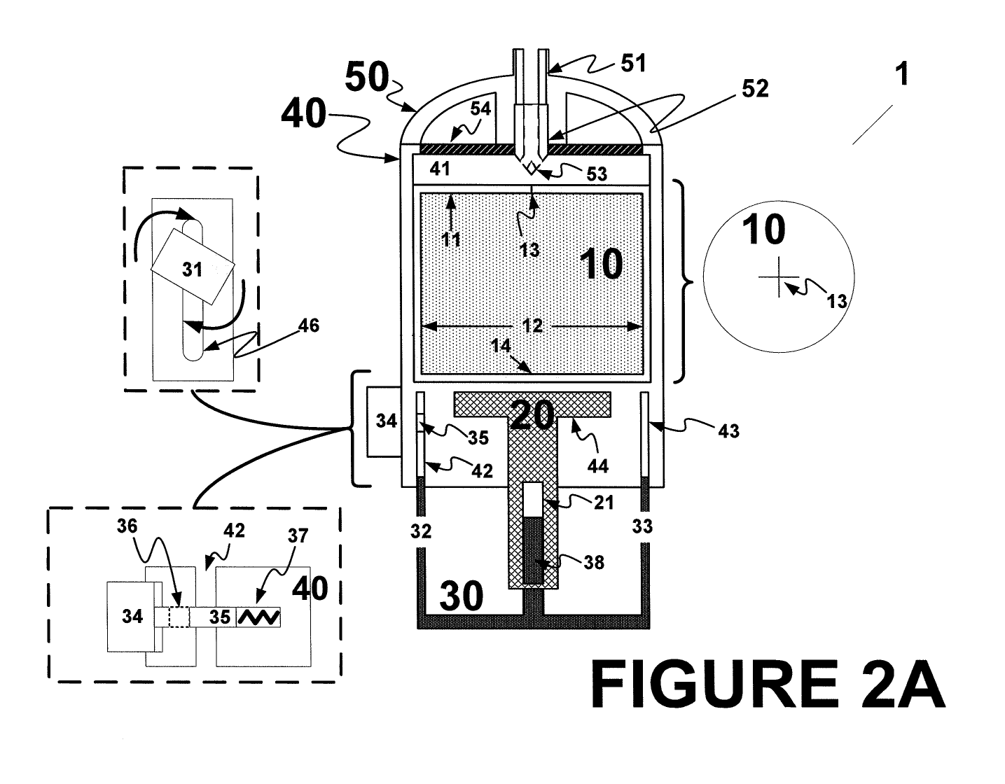

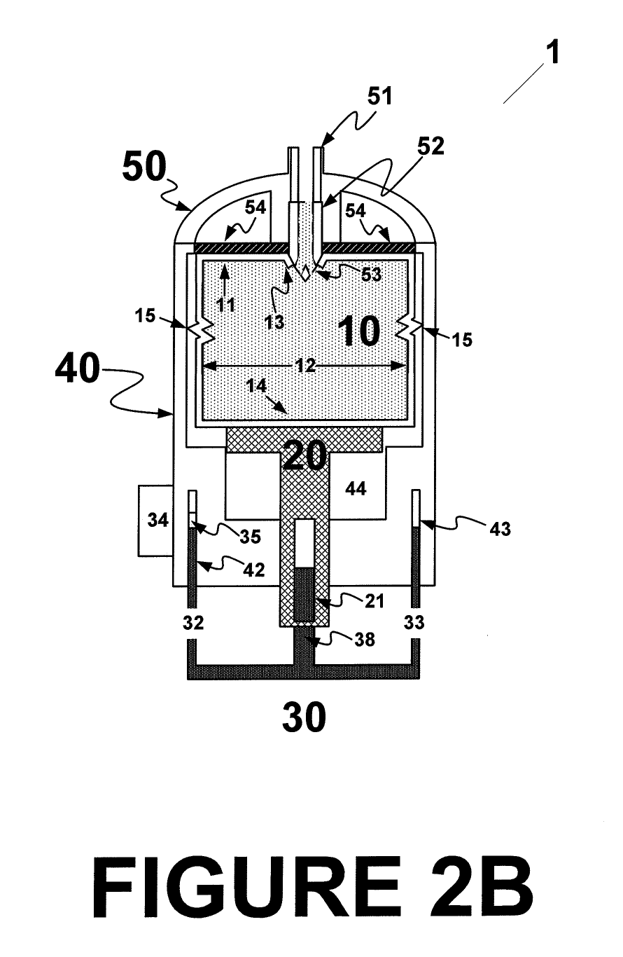

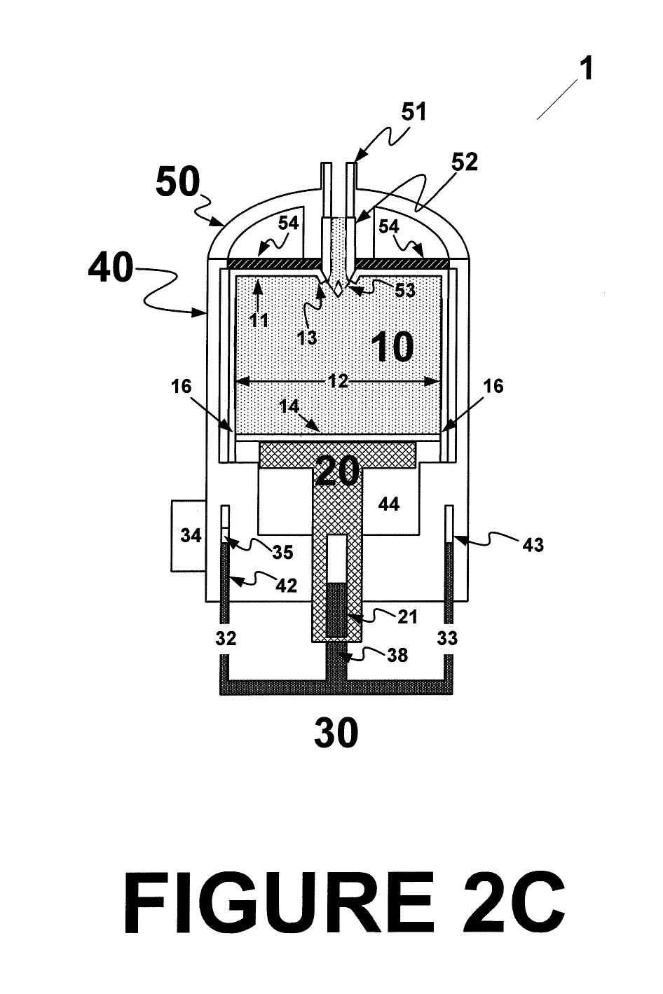

As shown in FIG. 2A, device 1 may be illustrated from a cross-sectional view. As illustrated, exemplary cartridge 10 may have a top portion 11 made up of a membrane 13 which may be pierced by cannula 53 of case 40. By piercing cartridge 10, cannula 53 allows medicament from cartridge 10 to flow through cannula 53 and into the channel 52 ultimately entering into medicament dispensing tip 51 of medicament dispenser 50.

Cannula 53 and channel 52 may be integrated with case 40 or may be part of interchangeable medicament dispenser 50. Channel 52 may be integrated with cannula 53 so that the two are one piece or they may be modular so that replacement of one can take place without replacement of the other. In an exemplary embodiment, cannula 53 may be interchangeable with medicament dispenser 50 to accommodate different types, styles, and designs of dispenser 50, tip 51, and/or cartridge 10. Alternatively, cannula 53 may remain part of case 40 and channel 52 may be interchangeable with medicament dispenser 50 to accommodate different types, styles, and designs of dispenser 50, tip 51, and/or cartridge 10.

In an exemplary embodiment, upon removal of cannula 53 from cartridge membrane 13, the oral suspension within cartridge 10 may be contained in a sterile environment for future dispensing. According to this exemplary embodiment, deformable portions of membrane 13 may allow for substantially tight sealing of medicament cartridge 10 so as to preserve medicament therein without losses during non-use of device 1.

An exemplary cartridge 10 may be made of any material suitable for puncturing and channeling fluid stored therein. An exemplary material may be plastic or silicone. An exemplary membrane 13 may be a crisscross slit formation in the outer surface 12 and inner surface 11 of cartridge 10 that may be moveably engaged by a pointed or other deflection inducing shaped cannula 53. Alternatively, membrane 13 may have any slit formation in the upper surface of cartridge 10 that permits a complementarily shaped cannula 53 to enter and exit without permanent deformation in the membrane 13 of cartridge 10.

In a preferred embodiment, membrane 13 of cartridge 10 may be configured to elastically deform when engaged by a complementary cannula 53 and return to a substantially closed configuration upon disengagement from cannula 53 so that medicament still within cartridge 10 remains therein after cannula 53 removal. Membrane 13 allows for the sterile containment of the oral suspension for future dispensing. The bottom of the cartridge 10 consists of a disc 14 spanning the girth 12 of the cartridge which is able to advance the medicament upon the application of pressure from the plunger mechanism 20. In an exemplary embodiment, disc 14 may advance upwardly within the outer walls of cartridge 10 to move medicament through the opening in membrane 13 in response to pressure from plunger mechanism 20. Alternatively disc 14 may be integrally molded with cartridge 10 to deform in conjunction with the outer walls of cartridge 10 to move medicament through the opening in membrane 13 in response to pressure from plunger mechanism 20. The top 11 of the cartridge 10 can be optionally removable allowing a user to refill the cartridge 10 with appropriate medicament.

Referring to the illustrative embodiment of FIG. 2A, in an unused state, device 1 may hold cartridge 10 in a repository 41 of case 40. Repository 41 may provide suitable space for displacement of cartridge 10 during dispensation so that suitable deflection of cartridge walls 11 and 12 may be achieved to allow medicament to enter cannula 53 when pressure is applied to the cartridge. Walls 54 may be part of case 40 or may optionally be part of interchangeable dispenser 50. Walls 54 may be sized and shaped to induce additional bending in cartridge wall 11 during dispensation. Alternatively, walls 54 may ensure sealing of case 40 to avoid loss of medicament during dispensation activities. In yet another alternative arrangement, walls 54 may act as coupling ports for interchangeable dispenser 50.

Further illustrated in FIG. 2A is plunger mechanism 20 and dosing mechanism 30 in an exemplary operable engagement. According to the illustrative embodiment of FIG. 2A, dosing mechanism 30 may be screwed into channel 21 of plunger mechanism 20 by adjuster component 38. While dosing mechanism 30 may be flat without walled edges 32 and 33 as shown in FIG. 1, the alternative dosing mechanism 30 of FIG. 2A may have arms 32 and 33 that engage slots 42 and 43, respectively, formed in case 40. According to this embodiment, as dosing mechanism 30 is pressed upwardly, causing plunger 20 to move from a resting position in plunger housing 44 of case 40, arms 32 and 33 of dosing mechanism 30 may slide up until they are prevented from doing so by lock combination of a latch 34 and bar 32.

According to an exemplary embodiment, latch 34 may act as an additional optimized dosing mechanism 30 to control the amount of medicament to be dispensed from cartridge 10. Alternatively, latch 34 may serve as a child lock to prevent dispensing medicament from cartridge 10. As illustrated in FIG. 2A, an exemplary latch 34 may be bolt-fastened so as to tightly resist movement along slot 46 in case 40. In this exemplary embodiment, latch 34 may resist interference from children who try to use device 1 or activate dosing mechanism 30 and/or plunger 20. In a locked position, bar 32 of latch 34 may preclude the arms 32/33 of the dosing mechanism 30 from progressing further into case 40 thereby preventing substantially any operative displacement of dosing mechanism 30 and/or plunger 20. Similar latches 31 may be used to restrict operative displacement of plunger 20 about channel 21 or other displacement points about device 1.

Alternatively, latch 34 may be a button that when compressed allows the arms 32/33 of dosing mechanism 30 to pass unimpeded within slot 42. For example, arm 32 of dosing mechanism 30 may be a thin cylindrical post that slidingly engages with case 40 slot 42. Latch 34 may be a spring loaded button that in an uncompressed state (as illustrated in FIG. 2A) provides bar 35 in the path of arm 32 of dosing mechanism 30 and the full length of case 40 slot 42. By compressing latch 34, bar 35 is compressed against a spring 37 until bypass 36 presents an opening in bar 35 for dosing mechanism 30 arm 32 to pass there-through. An exemplary spring 37 may be resilient enough to prevent children from activating device 1 to provide a medicament dose. Alternative child safety features for dosing devices such as device 1 known to those skilled in the art may also be incorporated.

As shown in FIG. 2A, tip 51 of dispensing mechanism 50 may couple to channel 52 to establish a substantially fluid contact between channel 52 and tip 51. Accordingly, medicament released from cartridge 10 through cannula 53 may travel through channel 52 into tip 51 and be dispensed. Tip 51 may be made of the same material as dispensing mechanism 50 or may be removable by way of screw or friction fitting. An exemplary dispensing mechanism 50 may be made of PVC or other durable plastic material.

Referring to FIG. 2B, a device 1 in an exemplary dispensing configuration may have dosing mechanism 30 in the desired dosing length configuration via screw adjustment between plunger channel 21 and adjuster component 38. Such a dosing mechanism 30 may be pressed upwardly into case 40 so that the arms 32/33 of dosing mechanism 30 traverse slots 42 and 43 within case 40 until they are precluded from further movement by bar 35 of latch 34 selectively placed according to a known dosing regimen. The pressing of dosing mechanism displaces plunger 20 from a resting position in cavity 44 of case 40. Plunger 20 applies pressure to the lower-most portion of cartridge 10 substantially about its girth 12.

Displacement of cartridge 10 may involve deformations 15 in cartridge 10 walls as well as deflections in membrane 13 at the points where cannula 53 engages membrane 13. As cannula 53 enters cartridge 10, medicament flows from cartridge 10 into channel 52 to be dispensed from tip 51. In this way, device 1 may dispense one or more different types of medicament from dispensing mechanism 50 to a desired recipient.

After dispensing a desired dose of medicament, dosing mechanism 30 may be withdrawn from case 40 thereby withdrawing plunger 20 from its compressed position against cartridge 10. Accordingly, cartridge 10 may retake its original shape for subsequent use or may be replaced by a new cartridge 10 containing the same or different medicament. According to an exemplary embodiment, cartridge 10 may be refillable and/or interchangeable. Thus, in one aspect, cartridge 10 may be a universal refillable cartridge that may enable users to refill the device 1 with a desired type of medicine.

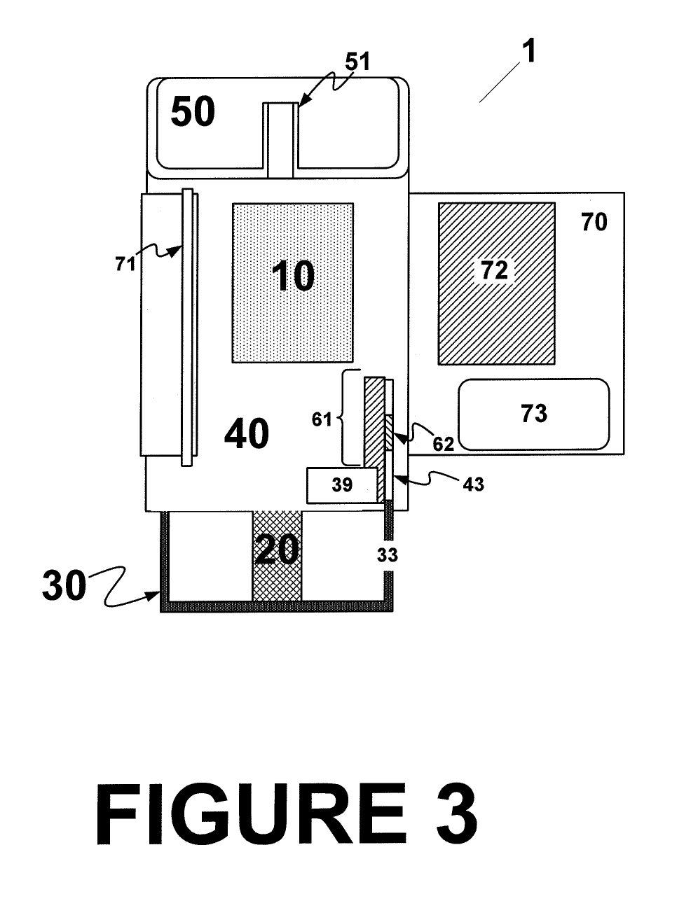

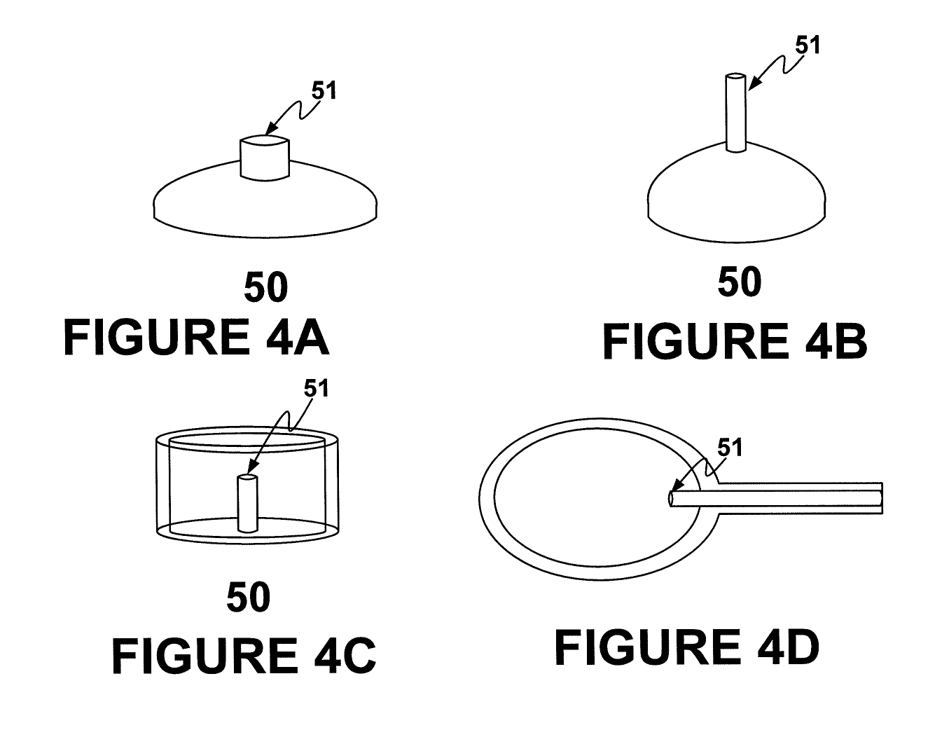

In a preferred embodiment, a cartridge 10 is filled with medicament and loaded into the encasement 40. An appropriate dispenser 50 is attached to the encasement 40. An appropriate dose may be looked up on a pullout dosing chart 70 based on one or more of a child's age, a child's weight, a child's age, and/or any other variable. While removing child safety lock 34 on dosing mechanism 30, dosing mechanism 30 is rotated to desired dosage. Child safety lock button 34 can now be released. Dispenser 50 is inserted into the child's mouth and the adult presses on dosing mechanism 30 which applies pressure on plunger mechanism 20 which applies pressure on disc 14 of cartridge 10 which advances dose into the dispenser 50 and into the mouth of the child. Alternatively, a cup shaped tip 51 (as illustrated in FIG. 3 and FIG. 4C) can be used which will allow user to measure and dispense the desired dose. The user will then place measured dose in another vessel such as a spoon for administering to the patient.

The dosing mechanism 30, plunger mechanism 20, and cartridge 10 interact together in the described sequence and are attached by pressure, snap, screw on, or any other method which insures safe operation of the design. The pull-out dosing chart 70 may wrap around the dosing mechanism 30, plunger mechanism 20, and cartridge 10, and together, the combination of the chart 70 and other components are secured by the encasement 40. The medicament dispenser 50 is then attached onto the encasement 40 by screw on, snap or any other method which ensures the safe operation of the design. The cap 60 attached to the encasement 40 over the dispenser/tip 50/51 by snap on, by screw on, or any other method which ensures the safe operation of the design.

Referring to FIG. 2C, a device 1 in an exemplary dispensing configuration may have dosing mechanism 30 in the desired dosing length configuration via screw adjustment between plunger channel 21 and adjuster component 38. Such a dosing mechanism 30 may be pressed upwardly into case 40 so that the arms 32/33 of dosing mechanism 30 traverse slots 42 and 43 within case 40 until they are precluded from further movement by bar 35 of latch 34 selectively placed according to a known dosing regimen. The pressing of dosing mechanism displaces plunger 20 from a resting position in cavity 44 of case 40. Plunger 20 applies pressure to disc 14 of cartridge 10.

Unlike cartridge 10 of the illustrative embodiment of FIG. 2B, cartridge 10 of the illustrative device embodiment of FIG. 2C may not have portions of its walls 15 undergoing deformation. Instead, disc 14 may have edges 16 which may be frictionally or slidingly engaged with the inner-most surfaces of the cartridge 10 walls so as to be moved upward by pressure of plunger mechanism 20. Disc 14 may displace medicament within cartridge 10 without necessitating deformation of cartridge 10 walls. An exemplary cartridge 10 according to the illustrative embodiment of FIG. 2C may be constructed of substantially rigid materials with the exception of the membrane 13. The bottom of the cartridge is open allowing for the plunger mechanism 20 to advance disc 14 into the interior of the cartridge.

Accordingly, an exemplary cartridge 10 of the type illustrated in FIGS. 2B and 2C may be used in any exemplary device 1 disclosed herein without changing the overall performance and objectives described. Those skilled in the art may recognize that device 1 may utilize a first cartridge 10 constructed so as to operate according to the illustrative embodiments of FIG. 2B and subsequently utilize a second cartridge 10 constructed so as to operate according to the illustrative embodiments of FIG. 2C. Preferably, device 1 may utilize a cartridge 10 constructed and operable according to the embodiments illustrated in FIG. 2C.

FIG. 3 illustrates an exterior view of medicament dispensing device 1 with a pull-out chart covering 70. According to FIG. 3, pullout chart adheres to case 40 by a spindle 71 engaged in the exterior surface of case 40 to allow for covering 70 to roll up or to just remain attached to case 40. Spindle 71 may be capable of being removed from case 40 to permit attachment of different coverings 70 depending on needs of the user. In an unraveled position, covering 70 may contain a transparent window 72 to permit viewing of medicament cartridge 10 in operation, for example, when covering 70 is wrapped about the outside of case 40. Additionally, covering 70 may also contain one or more sections 73 containing information for dosing, calibrating plunger 20 and/or dosing mechanism 30, and any such other information for dispensing medicament to the intended recipient.

As illustrated in FIG. 3, medicament dispensing portion 50 is in the shape of a cup whereby dispensation of medicament from tip 51 fills the cup 50 for further measuring or other form of administration.

Further illustrated in FIG. 3 is an alternative dosing mechanism 30 with dial control 39 and rack and pinion set 61/62. As dial 39 is turned on the external surface of case 40, an interior gear 61 is rotated by dial 39. The rotation of gear 61 rotates gear 62 so as to allow gear 62 to traverse slot 43 of case 40. Translational displacement of gear 62 within slot 43 provides for incremental control of the extent of dosing displacement of dosing mechanism 30 armature 33. In this way, the arm 33 of dosing mechanism will only displace the requisite distance as defined by precision control of dial 39. Dial 39 may contain markings or other indicia that describe the amount of revolutions to obtain a desired dose. Instructions for such use of dial 39 may be provided for on cover 70, for example, in information section 73.

Alternatively, rack and pinion set 61/62 may be visible to the viewer and be accompanied by markings on case 40 showing precisely where gear 62 should be located to achieve the desired dose. In this way, device 1 may provide precision dosing mechanisms via dosing mechanism 30 while providing easy access to dosing information and instructions via chart 70. The dosing control described with respect to FIG. 3 may be used in conjunction with any known cartridge 10 design, including those described and illustrated with respect to FIGS. 2B and 2C.

FIGS. 4A, 4B, 4C, and 4D illustrate some exemplary medicament dispensers 50 that may be used with device 1. FIG. 4A illustrates a nipple tip 51. FIG. 4B illustrates a dropper-type tip 51. FIG. 4C illustrates a cup-style tip 51. FIG. 4D illustrates a spoon-type tip 51.

The advantages medicament dispensing tips of the illustrative embodiments of FIGS. 4A and 4B provide for easy use with young children or pets which are accustomed to bottles and droppers for receiving other fluids. Advantages for medicament dispensing tips 51 of the type in the illustrative embodiment of FIG. 4C is to allow subsequent handling of medicament dispensed into a cup or to provide the medicament in a way that reduces risk of loss or inaccurate placement by means of a dropper or nipple. While only exemplary, the cup tip 51 of FIG. 4C may be in the shape of a cone, a cup with handle, or a child's sip cup.

The advantage of a medicament dispensing tip of the type of the illustrative embodiment of FIG. 4D includes the capability of directly providing accurate doses to an individual without concern for whether the spoon or holder used is representative of the desired dose. In other words, the spoon tip 51 of FIG. 4D avoids the concern that one is not using an actual teaspoon or table spoon and instead provides the proper dose according to the controlled dosing mechanisms herein disclosed. An exemplary spoon tip 51 may be any sized spoon known to those skilled in the art.

Each of the components of the medicament dispensing device 1 may be constructed of any suitable material fit for the purposes described. However, plastic components are preferable to avoid rusting and for easy cleaning/dishwasher safe, including plastic cover 70. Plastic cover 70 may be water resistant, as well as the internal moving components of case 40. Where metal parts must be used, water resilient metal components are preferable, but not necessary for operation of the various disclosed embodiments.

While the device and dispensing method have been described by way of example embodiments, it is understood that the words which have been used herein are words of description, rather than words of limitation. Changes may be made, within the purview of the appended claims without departing from the scope and spirit of the system and method in their broader aspects. Although the device and method have been described herein with reference to particular interrelated structures, interrelated materials, and interrelated embodiments, it is understood that the system and method is not limited to the particulars disclosed.

What I claim is:

1. A device, comprising:
a case having an upper portion, a lower portion, and a central portion;
a plunger having a head and a stem, the head of the plunger disposed between the central portion and the lower portion, and the stem disposed in the lower portion, wherein the head is configured to contact a cartridge containing medicament receivable within the central portion upon application of force to the stem;
a dosing assembly coupled to the stem and disposed partially within the case, the dosing assembly comprising a control system for selectively inhibiting displacement of the dosing assembly and plunger by precluding displacement of the dosing assembly within the case; and,
a customizable dispensing assembly coupled to the upper portion and configured to receive medicament from the cartridge upon application of force from the stem, the dispensing assembly configured to receive the medicament through an opening from the central portion to the upper portion.

2. The device of claim 1, further comprising a covering coupled to the case and configured to wrap about a portion of the case.

3. The device of claim 1, wherein the case is cylindrical.

4. The device of claim 1, wherein the dosing assembly is coupled to the stem through a screw-type attachment.

5. The device of claim 1, wherein the control system comprises a button and spring mechanism whereby depression of the button allows displacement of the dosing assembly.

6. The device of claim 1, wherein the control system comprises a dial and a rack and pinion assembly.

7. The device of claim 1, wherein the shape of the customizable dispensing assembly may be selected from the group consisting essentially of a nipple, a dropper, a cup, or a spoon.

8. The device of claim 1, wherein the control system comprises a screw and fastener combination.

9. The device of claim 2, wherein the covering permits viewing within the central portion.

10. The device of claim 2, wherein the covering contains medicament dispensing instructions.

11. A method of administering medicament, comprising the steps of:
coupling a dispensing tip to a case based on a medicament to be dispensed;
setting a control system to inhibit displacement of a dosing assembly within the case containing a cartridge of the medicament to be dispensed, the cartridge being disposed beneath the dispensing tip;
pressing the dosing assembly while it is coupled to a stem of a plunger which is disposed partially within the case;
displacing the plunger so that it displaces the bottom of the cartridge toward the dispensing tip and thereby causes the cartridge to provide medicament from the cartridge and into the dispensing tip;
dispensing medicament from the dispensing tip; and
inhibiting further displacement of the plunger by the control system.

12. The method of claim 11, wherein the step of coupling involves selecting the dispensing tip from the group consisting essentially of a nipple, a dropper, a cup, or a spoon.

13. The method of claim 11, wherein the step of setting the control system comprises mechanically limiting the ability of the dosing assembly to cause displacement of the plunger.

14. The method of claim 13, wherein mechanically limiting the ability of the dosing assembly to cause displacement of the plunger includes extending the dosing assembly a predetermined maximum distance from the stem of the plunger.

15. The method of claim 13, wherein mechanically limiting the ability of the dosing assembly to cause displacement of the plunger includes unfastening a latch, moving the latch within a slot in the case, and tightening the latch at the position along the slot that provides the maximum predetermined dosing assembly displacement.

16. The method of claim 13, wherein mechanically limiting the ability of the dosing assembly to cause displacement of the plunger includes turning a dial, turning a first gear as a result of turning the dial, turning a second gear as a result of turning the first gear, wherein turning of the first gear causes the second gear to limit the maximum displacement of the dosing assembly.

17. The method of claim 1, wherein the step of setting a control system further comprises setting the control system according to instructions on a chart coupled to the case.

18. The method of claim 1, further comprising installing a second cartridge of a different medicament into the case.

19. The method of claim 18, wherein the step of installing a second cartridge further comprises the step of coupling a second dispensing tip.

20. The method of claim 18, wherein the step of installing a second cartridge further comprises resetting the control system to inhibit the displacement of the dosing mechanism.
